(19) United States
(12) Patent Application Publication
(10) Pub. No.: US 2020/0390841 A1
(43) Pub. Date: Dec. 17, 2020

(54) COMPOSITIONS AND METHODS FOR TREATING WOUNDS

(71) Applicant: Canopy Medicine, Inc., Warwick, RI (US)

(72) Inventors: Beth ZIELINSKI-HABERSHAW, North Kingstown, RI (US); Jesse SPARTICHINO, Cambridge, MA (US)

(21) Appl. No.: 16/900,507

(22) Filed: Jun. 12, 2020

Related U.S. Application Data
(60) Provisional application No. 62/860,607, filed on Jun. 12, 2019.

(51) Int. Cl. A61K 36/74; A61K 36/38; A61Q 17/04; A61K 9/00; A61P 17/02; A61K 8/9789

(52) U.S. Cl. CPC A61K 36/74; A61K 36/38; A61K 8/9789; A61K 9/0014; A61P 17/02; A61Q 17/04

(57) ABSTRACT

The present invention relates to compositions containing combination of bark resin extracts from Vismia spp. and Calycophyllum spp. for treating skin wounds. Combination of bark resin extracts from Vismia spp. and Calycophyllum spp. inhibits inflammation, and induces wound contracture, epidermal keratinocyte migration and wound closure, and is thereby useful for treating wounds.

### FIELD OF THE INVENTION

This invention relates to formulations containing a combination of bark resin extracts from Vismia spp. and Calycophyllum spp., methods of making the formulations, and methods of using the formulations in treating skin wounds.

### BACKGROUND OF THE INVENTION

Wound healing is a complex and highly regulated process in which skin and underlying tissues repair themselves after injury. Rapid wound closure is imperative to prevent tissue destruction, potential infection and eventual loss of tissue function. TNF-α plays a key role in the course of healthy wound healing, and dysregulation in the level of this cytokine can inhibit the normal process of wound healing, resulting in non-healing chronic wounds. There is a critical need, worldwide, for topical treatments that would correct the imbalance of TNF-α at the wound sites of patients experiencing chronic inflammation, ulcerations and lack of wound closure.

### SUMMARY OF THE INVENTION

Featured herein are compositions containing combination of bark resin extracts from one or more plants of the genus Vismia and one or more plants of the genus Calycophyllum and use thereof in treating wounds (e.g., chronic wounds and acute wounds). Depending upon the ratio of bark resin extracts of the genus Vismia and the genus Calycophyllum, aspects of the wound healing process may be modulated or controlled. For example, in the case of chronic wounds, increasing the ratio of Vismia extract to Calycophyllum extract may result in significant reductions in inflammation and wound closure following extract-induced decreases in local TNF-α concentrations and allowance for epidermal keratinocyte mitosis and wound closure. Conversely, in the case of acute wounds and avoidance of epithelial scarring, increasing the ratio of Calycophyllum extract to Vismia extract may allow for more moderate, extract-induced reductions in inflammation, avoidance of bacterial and fungal colonization, decreases in the rate of epidermal keratinocyte mitosis and avoidance of fibrosis.

A first aspect features a composition (e.g., a pharmaceutical composition) containing about 0.001-50% (e.g., about 0.001-50%, 0.001-40%, 0.001-30%, 0.001-20%, 0.001-10%, 0.001-1%, 0.01-50%, 0.01-40%, 0.01-30%, 0.01-20%, 0.01-10%, 0.01-1%, 0.1-50%, 0.1-40%, 0.1-30%, 0.1-20%, 0.1-10%, or 0.1-1%) (w/v) bark resin extract (e.g., aqueous bark resin extract) from a plant of genus Vismia (e.g., Vismia angusta) and about 0.001-50% (e.g., about 0.001-50%, 0.001-40%, 0.001-30%, 0.001-20%, 0.001-10%, 0.001-1%, 0.01-50%, 0.01-40%, 0.01-30%, 0.01-20%, 0.01-10%, 0.01-1%, 0.1-50%, 0.1-40%, 0.1-30%, 0.1-20%, 0.1-10%, or 01-1%)(w/v) bark resin extract (e.g., aqueous bark resin extract) from a plant of genus Calycophyllum (e.g., Calycophyllum spruceanum).

In some embodiments of the above aspect, the ratio of the bark resin extract from the plant of genus Vismia to the bark resin extract from the plant of genus Calycophyllum is 1:1. In other embodiments, the ratio of the bark resin extract from the plant of genus Vismia to the bark resin extract from the plant of genus Calycophyllum is more than 1:1 (e.g., 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, or more). In alternative embodiments, the ratio of the bark resin extract from the plant of genus Vismia to the bark resin extract from the plant of genus Calycophyllum is less than 1:1 (e.g., 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:21, 1:22, 1:23, 1:24, 1:25, 1:26, 1:27, 1:28, 1:29, 1:30, 1:31, 1:32, 1:33, 1:34, 1:35, 1:36, 1:37, 1:38, 1:39, 1:40, 1:41, 1:42, 1:43, 1:44, 1:45, 1:46, 1:47, 1:48, 1:49, 1:50, or less).

In some embodiments of the above aspect, the pharmaceutical composition includes at least 50% (v/v) water. In some embodiments, the pharmaceutical composition further includes a pharmaceutically acceptable carrier, excipient, or vehicle. In further embodiments, the pharmaceutical composition includes one or more antibiotic agents, antiseptic agents, antifungal agents, antiviral agents, analgesic agents, UV-absorbing agents, and/or moisturizing agents.

In some instances, the pharmaceutical composition is formulated as a topical formulation. In further embodiments, the pharmaceutical composition is formulated as a lotion, cream, salve, liniment, ointment, gel, paste, tonic, unguent, spray, soap, shampoo, or lip balm.

Also featured herein is a method of treating a wound, such as a chronic wound (e.g., a venous ulcer, a diabetic ulcer, a pressure ulcer, or rash associated with shingles, psoriasis, allergy (e.g., induced by irritants, such as certain chemical, plants, animals, etc.), or dermatitis) or an acute wound (e.g., a trauma wound, a burn wound, or a surgical wound) in a subject (e.g., a human) in need thereof, by administering to the subject a therapeutically effective amount of the pharmaceutical composition of the first aspect. Also described herein is the pharmaceutical composition of the first aspect for use in treating a wound, such as a chronic wound (e.g., a venous ulcer, a diabetic ulcer, a pressure ulcer, or rash associated with shingles, psoriasis, allergy (e.g., induced by irritants, such as certain chemical, plants, animals, etc.), or dermatitis) or an acute wound (e.g., a trauma wound, a burn wound, or a surgical wound) in a subject (e.g., a human) in need thereof.

In some embodiments, a chronic wound in a subject is treated by administering to the subject a therapeutically effective amount of the pharmaceutical composition of the first aspect, wherein the ratio of the bark resin extract from the plant of genus Vismia to the bark resin extract from the plant of genus Calycophyllum is 1:1 or is more than 1:1 (e.g., 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, or more). In some embodiments, an acute wound in a subject is treated by administering to the subject a therapeutically effective amount of the pharmaceutical composition of the first aspect, wherein the ratio of the bark resin extract from the plant of genus Vismia to the bark resin extract from the plant of genus Calycophyllum is 1:1 or is less than 1:1 (e.g., 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:21, 1:22, 1:23, 1:24, 1:25, 1:26, 1:27, 1:28, 1:29, 1:30, 1:31, 1:32, 1:33, 1:34, 1:35, 1:36, 1:37, 1:38, 1:39, 1:40, 1:41, 1:42, 1:43, 1:44, 1:45, 1:46, 1:47, 1:48, 1:49, 1:50, or less).

In some embodiments, the pharmaceutical composition is administered to the subject for 15 days or less (e.g., 10 days, 7 days, or less). In some embodiments, the pharmaceutical composition reduces inflammation, induces wound contracture, induces wound closure, reduces release of TNF-α from peripheral blood mononuclear cells, and/or induces migration of epidermal keratinocytes.

### Definitions

As used herein, the term “administering” refers to the act of providing or giving a subject a therapeutic composition (e.g., a composition containing a combination of bark resin extracts from Vismia spp. and Calycophyllum spp.) or agent (e.g., one or more additional agents described herein, such as antibiotic agents, antiseptic agents, antifungal agents, antiviral agents, analgesic agents, UV-absorbing agents, and moisturizing agents), by any effective route (e.g., topically). Exemplary routes of administration are described herein below.

The term “effective amount” or “therapeutically effective amount,” as used herein, means an amount of a composition of the present invention, that, when administered to a subject (e.g., a human subject or an animal model) in need of such treatment, is sufficient to effect treatment, as defined herein.

For example, an effective amount or therapeutically effective amount of a composition containing a combination of bark resin extracts from Vismia spp. and Calycophyllum spp., when administered to a subject (e.g., a human subject or an animal model), is sufficient to treat wound, induce wound healing, induce wound contracture, induce wound closure, and reduce inflammation (e.g., reduce release of inflammatory cytokines (e.g., TNF-α, IFNγ, IL-1, IL-6, IL-1β, IL-12, IL-18, etc.)).

The term “pharmaceutical composition,” or “pharmaceutical formulation,” as used herein, means a combination of a composition of the invention, and a carrier, diluent, excipients, solvent, and/or adjuvants that are compatible with composition of the present invention, and is not deleterious to the recipient thereof. Exemplary methods for preparing pharmaceutical compositions are known to those of ordinary skill in the art and described herein below.

The terms “treat”, “treating”, and “treatment” refer to any treatment of a wound in a subject, such as a mammal, particularly a human, by the compositions described herein (e.g., composition containing a combination of bark resin extracts from Vismia spp. and Calycophyllum spp.) and include: reducing inflammation (e.g., reduce release of inflammatory cytokines (e.g., TNF-α, IFNγ, IL-1, IL-6, IL-1β, IL-12, IL-18, etc.), inducing wound contracture, inducing wound closure, and inducing wound healing, without addressing the underlying disease or condition (e.g., treats diabetic ulceration without addressing the underlying disease, diabetes).

As used herein, the terms “increase,” “increasing,” “induce” or “inducing” and “decrease,” “decreasing,” “reduce” or “reducing” refer to modulating resulting in, respectively, greater or lesser amounts, of function, expression level, occurrence, or activity of a metric relative to a reference. For example, subsequent to administration of the composition described herein (e.g., composition containing a combination of bark resin extracts from Vismia spp. and Calycophyllum spp.), TNF-α release by cells at the site of wound may reduce or decrease by at least 5% or more (e.g., between 5-20%, between 5-50%, between 10-50%, between 10-80%, between 20-80%, or between 20-100% (e.g., 5%, 10%, 15%, 20%, 25%, 30%, 40%, 50%, 60%, 70%, 80%, 90%, 95%, 99%, or more)) relative to TNF-α release prior to administration of the composition. Furthermore, subsequent to administration of the composition described herein (e.g., composition containing a combination of bark resin extracts from Vismia spp. and Calycophyllum spp.), healing and/or closure of wound in a subject may be induced or increased by at least 5% or more (e.g., between 5-20%, between 5-50%, between 10-50%, between 10-80%, between 20-80%, or between 20-100% (e.g., 5%, 10%, 15%, 20%, 25%, 30%, 40%, 50%, 60%, 70%, 80%, 90%, 95%, 99%, or more)), such that healing and/or closure of wound is at least 5% (e.g., between 5-20%, between 5-50%, between 10-50%, between 10-80%, between 20-80%, or between 20-100% (e.g., 5%, 10%, 15%, 20%, 25%, 30%, 40%, 50%, 60%, 70%, 80%, 90%, 95%, 99%, or more)) faster relative to healing and/or closure of wound prior to administration of the composition, or compared to wound in a reference subject that is not treated with the composition. Generally, the metric is measured subsequent to administration at a time that the administration has had the recited effect, e.g., at least 3 hours, 6 hours, 12 hours, 18 hours, 1 day, 2 days, 3 days, 4 days, 5 days, 6 days, 7 days, 1 week, 2 weeks, 3 weeks, 4 weeks, 5 weeks, 6 weeks, 7 weeks, 8 weeks, 3 months, 4 months, 5 months, or 6 months, after a treatment regimen has begun. The term “reducing” is used interchangeably with the term “decreasing” herein. The term “increasing” is used interchangeably with the term “inducing” herein.

The terms “composition of the present invention,” “composition of the invention,” “composition featured herein,” or “featured composition” refer to compositions containing a combination of bark resin extracts from Vismia spp. and Calycophyllum spp. in concentrations, ratios and formulations detailed in the present disclosure. These terms also refer to pharmaceutical formulations prepared with these compositions. Such compositions include a composition containing 0.001-50% (w/v) bark resin extract of Vismia angusta and 0.001-50% (w/v) bark resin extract of Calycophyllum spruceanum.

The terms “comprising” and “including” as used herein, are used in their open, non-limiting sense.

### BRIEF DESCRIPTION OF THE DRAWINGS

FIG. 1 is a graph showing TNF-α release from human peripheral blood mononuclear cells (PBMCs) that were incubated for 12 hours in medium alone (negative control, N), medium+LPS (positive control, P), medium+LPS+Vismia angusta bark resin extract (VA), medium+LPS+Calycophyllum spruceanum bark resin extract (CA), or medium+LPS+Vismia angusta bark resin extract+Calycophyllum spruceanum bark resin extract (VACA).

FIG. 2 shows micrographs of epidermal keratinocytes from an in vitro scratch assay. Micrographs on the top panel show control epidermal keratinocytes at 0 hour (t=0; top left), control epidermal keratinocytes at 0 hour following the infliction of wound (t=0; top middle), and control epidermal keratinocytes at 2 weeks following the infliction of wound (t=2 weeks; top right). Micrographs on the bottom panel show epidermal keratinocytes, following the infliction of wound, that were incubated for 2 weeks (t=2 weeks) in 10% w/v Calycophyllum spruceanum bark resin extract (bottom left), 10% w/v Vismia angusta bark resin extract (bottom middle), and 10% w/v Calycophyllum spruceanum combined with 10% w/v Vismia angusta bark resin extract (bottom right).

FIG. 3 shows images of an insect bite-associated dermal ulceration in a human before treatment (pre-treatment) and after treatment (on days 1, 2, 3, 4, 7, and 8) with a composition containing 10% (w/v) Vismia angusta bark resin extract and 10% (w/v) Calycophyllum spruceanum bark resin extract.

FIG. 4 shows images of shingles rash in a human before treatment (pre-treatment) and after treatment (days 1 and 2) with a composition containing 10% (w/v) Vismia angusta bark resin extract and 10% (w/v) Calycophyllum spruceanum bark resin extract.

### DETAILED DESCRIPTION

Wound healing is a complex process involving four distinct stages: hemostasis, acute inflammation, granulation/proliferation, and remodeling. Upon injury to epidermal and dermal tissues, primary hemostasis occurs that involves platelet aggregation and activation, and formation of a platelet plug to seal the wound and initiate blood clotting. Secondary hemostasis, a complex coagulation cascade, occurs simultaneously with the activation of blood clotting factors, such as Factor VII, Factor X and prothrombin. Prothrombin is activated to thrombin that interacts with fibrinogen to form a fibrin clot, which ultimately prevents blood flow from the injured tissues. This process typically occurs between minutes to hours of the initial injury. The second stage of wound healing, acute inflammation, can last up to 20 days following hemostasis and involves the biochemical synthesis and bioactivity of hundreds of cytokines, importantly TNF-α, as well as chemokines and matrix metalloproteinases. During this stage, macrophages attempt to engulf damaged cells, foreign debris and bacteria. Neutrophils extravasate from surrounding capillaries and amplify the phagocytic response. Surrounding capillaries also become dilated and their walls become permeable, thus allowing additional immune cells and fluid from plasma to accumulate in the wound area. The entire response is characterized by heat, redness, swelling and pain. The third stage of wounding healing is the formation of granulation tissue and proliferation. This stage does not begin at a discrete time and may begin as early as 1 week and up to 3 weeks following injury. Connective tissue cells, fibroblasts, begin to proliferate and synthesize collagen type III, thus laying down a matrix for further closure of the wound. Neovascularization occurs through both angiogenesis, which is the formation of new blood vessels from existing blood vessels and vasculogenesis, which is formation of new vessels from endothelial progenitor cells. Growth factors, are secreted, promoting fibroblast and epidermal keratinocyte migration and proliferation. This results in tissue contracture and bridging of the wound edges in preparation for the final phase of wound healing, which is tissue remodeling. The fourth and final stage of wounding healing can begin as early as 3 weeks following injury and last up until 12 months post-injury. This final stage is characterized by further epidermal keratinocyte migration. Collagen III is remodeled to collagen I. Additionally, collagen is aligned along tension lines and excess fluids are reabsorbed.

Many factors can inhibit the normal process of wound healing resulting in a stage of chronic inflammation and ulceration. Patient immune and disease status, infection and nutrition are factors that play important roles in determining the ability of wounds to heal properly. TNF-α plays a key role in the course of healthy wound healing. TNF-α is a pro-inflammatory cytokine that induces immune cell activation, phagocytosis and chemoattraction of systemic immune cells. Level of TNF-α is significant during acute inflammation and must decrease during the granulation and proliferation phase for wound healing to continue normally. Chronically elevated levels of TNF-α lead to persistent inflammation and ulceration of the wound area. Studies have shown that patients with chronic dermal wounds and ulcerations exhibit high levels of TNF-α that exacerbate inflammation leading to chronic inflammation and ulceration. Hence, topical treatments that would correct the imbalance of TNF-α (e.g., reduce TNF-α) quickly at the wound sites of patients with chronic wound is of utmost clinical importance.

As indigenous medicines, Vismia bark resin and Calycophyllum bark resin have been used, independently, to support wound closure and as an anti-infective, respectively. The present disclosure describes a combination of bark resin extracts from both Vismia spp. and Calycophyllum spp. that, for the first time, demonstrates the ability to reduce TNF-α release from LPS-stimulated human PBMCs in vitro, and heal open wounds associated with shingles within 5 days and chronic ulceration within 7 days in human subjects. Thus, the composition described herein is useful for treating chronic and acute dermal wounds in human.

Vismia Spp. and Calycophyllum Spp. Bark Resin Extract

Featured in the disclosure are compositions or formulations containing a combination of bark resin extracts from Vismia spp. and Calycophyllum spp. For the purposes described herein, bark resin extracts from Vismia spp. and Calycophyllum spp. can be prepared in water. For example, aqueous bark resin extract from plants of the genus Vismia may be combined with aqueous bark resin extract from plants of the genus Calycophyllum for obtaining the compositions featured herein.

Vismia is a genus of flowering plants in the family Hypericaceae. Members of the genus Vismia are small trees and shrubs found in tropical and subtropical areas of Africa, Central America, and South America. Species of the genus Vismia include Vismia affinis Oliv., Vismia atlantica L. Marinho & M. V. Martins, Vismia baccifera (L.) Planch. & Triana, Vismia bemerguii M. E. Berg, Vismia billbergiana Beurl., Vismia brasiliensis Choisy, Vismia camparaguey Sprague & L. Riley, Vismia cauliflora A. C. Sm., Vismia cavalcantei Van den Berg, Vismia cavanillesiana Cuatrec., Vismia cayennensis (Jacq.) Pers., Vismia confertiflora Spruce ex Reichardt, Vismia corymbosa A. Chev., Vismia crassa (Rusby) S. F. Blake, Vismia cuatrecasasii Ewan, Vismia ferruginea Kunth, Vismia floribunda Sprague, Vismia glabra Ruiz & Pav., Vismia gracilis Hieron., Vismia guianensis (Aubl.) Pers., Vismia guineensis (L.) Choisy, Vismia japurensis Rchb.f., Vismia jefensis N. Robson, Vismia laevis Planch. & Triana, Vismia lateriflora Ducke, Vismia latifolia (Aubl.) Choisy, Vismia latisepala N. Robson, Vismia laurentii De Wild., Vismia lauriformis (Lam.) Choisy, Vismia laxiflora Rchb.f., Vismia lindeniana Decne. ex Turcz., Vismia angusta Miq. (synonym: Vismia macrophylla Kunth), Vismia magnoliifolia Cham. & Schltdl., Vismia mandurr Hieron., Vismia martiana Rchb.f., Vismia micrantha A. St.-Hil., Vismia minutiflora Ewan, Vismia obtusa Spruce ex Reichardt, Vismia orientalis Engl., Vismia parviflora Cham. & Schltdl., Vismia pauciflora Milne-Redh., Vismia pentagyna (Spreng.) Ewan, Vismia plicatifolia Hochr., Vismia pozuzoensis Engl., Vismia ramuliflora Miq., Vismia rubescens Oliv., Vismia rufa Cuatrec., Vismia rusbyi Ewan, Vismia sandwithii Ewan, Vismia sessilifolia (Aubl.) Choisy, Vismia sprucei Sprague, Vismia steyermarkii N. Robson, Vismia tenuinervia (M. E. Berg) N. Robson, Vismia tomentosa Rui, and Vismia torrei Mendes.

Bark resin extract (e.g., aqueous bark resin extract) from Vismia spp. to be included in the composition featured herein can be bark resin extract (e.g., aqueous bark resin extract) from one or more species of the genus Vismia (e.g., one or more species of Vismia mentioned hereinabove). For example, bark resin extract (e.g., aqueous bark resin extract) from Vismia spp. to be included in the composition featured herein can be bark resin extract (e.g., aqueous bark resin extract) from Vismia angusta.

The composition featured herein may contain about 0.001-50% (e.g., about 0.001-50%, 0.001-40%, 0.001-30%, 0.001-20%, 0001-10%, 0001-1%, 0.01-50%. 0.01-40%, 0.01-30%, 0.01-20%, 0.01 10%, 0.01-1%, 0.1-50%, 0.1-40%, 0.1-30%, 0.1-20%. 0.1-10%, or 0.1-1% (e.g., about 0.001%, 0.005%, 0.01%, 0.05%, 0.1%, 0.5%. 1%, 5%, 10%, 15%, 20%, 25%. 30%, 35%, 40%, 45%, or 50%)) (w/v) bark resin extract (e.g., aqueous bark resin extract) from one or more species of the genus Vismia (e.g., one or more species of Vismia mentioned hereinabove, such as Vismia angusta). For example, composition featured herein may contain about 0.001-50% (e.g., about 0.001-50%, 0.001-40%, 0.001-30%, 0.001-20%, 0.001-10%, 0.001-1%, 0.01-50%, 0.01-40%, 0.01-30%, 0.01-20%, 0.01-10%, 0.01-1%, 0.1-50%, 01-40%, 01-30%, 0.1-20%, 0.1-10%, or 0.1-1% (e.g., about 0.001%, 0.005%, 0.01%, 0.05%, 0.1%, 0.5%, 1%, 5%, 10%, 15%, 20%, 25%, 30%, 35%, 40%, 45%, or 50%)) (w/v) bark resin extract (e.g., aqueous bark resin extract) from Vismia angusta.

Calycophyllum is a genus of flowering plants in the family Rubiaceae. It was described by Augustin Pyramus de Candolle in 1830. Members of the genus Calycophyllum are found in Mexico, Central America, South America and the West Indies. Species of the genus Calycophyllum include Calycophyllum candidissimum (Vahl) DC. (common names: Lemonwood, Digame Lancewood), Calycophyllum intonsum Steyerm., Calycophyllum megistocaulum (K. Krause) C. M. Taylor., Calycophyllum merumense Steyerm., Calycophyllum multiflorum Griseb., Calycophyllum obovatum (Ducke) Ducke., Calycophyllum papillosum J. H. Kirkbr., Calycophyllum spectabile Steyerm., Calycophyllum spruceanum (Benth.) Hook.f. ex K. Schum., Calycophyllum tefense J. H. Kirkbr., and Calycophyllum venezuelense Steyerm.

Bark resin extract (e.g., aqueous bark resin extract) from Calycophyllum spp. to be included in the composition featured herein can be bark resin extract (e.g., aqueous bark resin extract) from one or more species of the genus Calycophyllum (e.g., one or more species of Calycophyllum mentioned hereinabove). For example, bark resin extract (e.g., aqueous bark resin extract) from Calycophyllum spp. to be included in the composition featured herein can be bark resin extract (e.g., aqueous bark resin extract) from Calycophyllum spruceanum (common name: capirona).

The composition featured herein may contain about 0.001-50% (e.g., about 0.001-50%, 0.001-40%, 0.001-30%, 0.001-20%, 0.0001-10%, 0.001-1%, 0.01-50%, 0.01-40%, 0.01-30%, 0.01-20%, 0.01-10%, 0.01-1%, 0.1-50%, 0.1-40%, 0.1-30%, 0.1-20%, 0.1-10%, or 0.1-1% (e.g. about 0.001%, 0.005%, 0.01%, 0.05%, 0.1%, 0.5%, 1%, 5%, 10%, 15%, 20%, 25%, 30%, 35%, 40%, 45%, or 50%)) (w/v) bark resin extract (e.g., aqueous bark resin extract) from one or more species of the genus Calycophyllum (e.g., one or more species of Calycophyllum mentioned hereinabove, such as Calycophyllum spruceanum). For example, composition featured herein may contain about 0.001-50% (e.g., about 0.001-50%, 0.001-40%, 0.001-30%, 0.001-20%, 0.001-10%, 0.001-1%, 0.01-50%, 0.01-40%, 0.01-30%, 0.01-20%, 0.01-10%, 0.01-1%, 0.1-50%, 0.1-40%, 0.1-30%, 0.1-20%, 0.1-10%, or 0.1-1% (e.g. about 0.001%, 0.005%, 0.01%, 0.05%, 0.1%, 0.5%, 1%, 5%, 10%, 15%, 20%, 25%, 30%, 35%, 40%, 45%, or 50%) (w/v) bark resin extract (e.g., aqueous bark resin extract) from Calycophyllum spruceanum.

In certain embodiments, the composition featured herein may contain a combination of about 0.001-50% (e.g., about 0.001-50%, 0.001-40%, 0.001-30%, 0.001-20%. 0.001-10%, 0.001-1%, 0.01-50%, 0.01-40%, 0.01-30%, 0.01-20%0.01-10%, 0.01-1%, 0.1-50%, 0.1-40%. 0.1-30%, 0.1-20%, 0.1-10%, or 0.1-1% (e.g., about 0.001%, 0.005%, 0.01%, 005%, 0.1%, 0.5%, 1%, 5%. 10%, 15%, 20%. 25%, 30%, 35%, 40%, 45%, or 50%)) (w/v) bark resin extract (e.g., aqueous bark resin extract) from one or more plants of the genus Vismia (e.g., one or more species of Vismia mentioned hereinabove, such as Vismia angusta) and about 0.001-50% (e.g., about 0001-50%. 0.001-40%, 0.001-30%, 0.001-20%, 0.001-10%, 0.001-1%, 0.01-50%, 001-40%, 0.01-30%, 01-20%, 0.01-10%, 0.01-1%, 0.1-50%, 0.1-40%, 0.1-30%, 0.1-20%, 0.1-10%, or 0.1-1% (e.g., about 0.001%, 0.005%, 0.01%, 0.05%, 0.1%, 0.5%, 1%, 5%, 10%, 15%, 20%, 25%, 30%, 35%, 40%, 45%, or 50%)) (w/v) bark resin extract (e.g., aqueous bark resin extract) from one or more plants of the genus Calycophyllum (e.g., one or more species of Calycophyllum mentioned hereinabove, such as Calycophyllum spruceanum). In particular, the composition featured herein may contain a combination of about 0.001-50% (e.g., about 0.001-50%, 0.001-40%, 0.001-30%, 0.001-20%, 0.001-10%, 0.001-1%, 0.01-50%, 0.01-40%, 0.01-30%, 0.01-20%, 0.01-10%, 0.01-1%, 0.1-50%, 0.1-40%, 0.1-30%, 0.1-20%, 0.1-10%, or 0.1-1% (e.g., about 0.001%, 0.005%, 0.01%, 0.05%, 0.1%, 0.5%, 1%, 5%, 10%, 15%, 20%, 25%, 30%, 35%, 40%, 45%, or 50%)) (w/v) bark resin extract (e.g., aqueous bark resin extract) from Vismia angusta and about 0.001-50% (e.g. about 0.001-50%, 0.001-40%, 0.001-30%, 0.0001-20%, 0.0001-10%, 0001-1%, 0.01-50%, 0.01-40%, 0.01-30%, 0.01-20%, 0.01-10%, 0.01%, 0150%, 0.1-0%, 01-30%, 0.1-20%, 0.1-10%, or 01-1% (e.g. about 0.001%, 0.005%, 0.01%, 0.05%, 0.1%, 0.5%, 1%, 5%, 10%, 15%, 20%, 25%, 30%, 35%, 40%, 45%, or 50%)) (w/v) bark resin extract (e.g., aqueous bark resin extract) from Calycophyllum spruceanum.

In some embodiments, in the composition featured herein, ratio of bark resin extract (e.g., aqueous bark resin extract) of one or more plants of the genus Vismia (e.g., one or more species of Vismia mentioned hereinabove, such as Vismia angusta) to bark resin extract (e.g., aqueous bark resin extract) of one or more plants of the genus Calycophyllum (e.g., one or more species of Calycophyllum mentioned hereinabove, such as Calycophyllum spruceanum) is 1:1. For example, the ratio of bark resin extract (e.g., aqueous bark resin extract) of Vismia angusta to bark resin extract (e.g., aqueous bark resin extract) of Calycophyllum spruceanum in the composition featured herein may be 1:1. In other embodiments, in the composition featured herein, ratio of bark resin extract (e.g., aqueous bark resin extract) of one or more plants of the genus Vismia (e.g., one or more species of Vismia mentioned hereinabove, such as Vismia angusta) to bark resin extract (e.g., aqueous bark resin extract) of one or more plants of the genus Calycophyllum (e.g., one or more species of Calycophyllum mentioned hereinabove, such as Calycophyllum spruceanum) is more than 1:1 (e.g., 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, or more (e.g., 10:1)). In particular, the ratio of bark resin extract (e.g., aqueous bark resin extract) of Vismia angusta to bark resin extract (e.g., aqueous bark resin extract) of Calycophyllum spruceanum in the composition featured herein may be more than 1:1 (e.g., 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, or more (e.g., 10:1)). For example, the ratio of bark resin extract (e.g., aqueous bark resin extract) of Vismia angusta to bark resin extract (e.g., aqueous bark resin extract) of Calycophyllum spruceanum in the composition featured herein may be 10:1. In alternative embodiments, in the composition featured herein, ratio of bark resin extract (e.g., aqueous bark resin extract) of one or more plants of the genus Vismia (e.g., one or more species of Vismia mentioned hereinabove, such as Vismia angusta) to bark resin extract (e.g., aqueous bark resin extract) of one or more plants of the genus Calycophyllum (e.g., one or more species of Calycophyllum mentioned hereinabove, such as Calycophyllum spruceanum) is less than 1:1 (e.g., 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:21, 1:22, 1:23, 1:24, 1:25, 1:26, 1:27, 1:28, 1:29, 1:30, 1:31, 1:32, 1:33, 1:34, 1:35, 1:36, 1:37, 1:38, 1:39, 1:40, 1:41, 1:42, 1:43, 1:44, 1:45, 1:46, 1:47, 1:48, 1:49, 1:50, or less (e.g., 1:10)). In particular, the ratio of bark resin extract (e.g., aqueous bark resin extract) of Vismia angusta to bark resin extract (e.g., aqueous bark resin extract) of Calycophyllum spruceanum in the composition featured herein may be less than 1:1 (e.g., 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:21, 1:22, 1:23, 1:24, 1:25, 1:26, 1:27, 1:28, 1:29, 1:30, 1:31, 1:32, 1:33, 1:34, 1:35, 1:36, 1:37, 1:38, 1:39, 1:40, 1:41, 1:42, 1:43, 1:44, 1:45, 1:46, 1:47, 1:48, 1:49, 1:50, or less (e.g., 1:10)). For example, the ratio of bark resin extract (e.g., aqueous bark resin extract) of Vismia angusta to bark resin extract (e.g., aqueous bark resin extract) of Calycophyllum spruceanum in the composition featured herein may be 1:10.

### Wounds

Compositions featured herein (e.g., a composition containing a combination of bark resin extracts (e.g., aqueous bark resin extracts) of one or more plants of the genus Vismia (e.g., Vismia angusta) and one or more plants of the genus Calycophyllum (e.g., Calycophyllum spruceanum)) may be used for treating wounds (e.g., dermal wounds) in a subject (e.g., a human subject or an animal model) in need thereof. The featured composition can be used for treating chronic wounds (e.g., chronic dermal wounds) and/or acute wounds (e.g., acute dermal wounds).

### Chronic Wounds

The compositions and methods described herein can be used for treating a chronic non-healing wound. A chronic wound (e.g., chronic dermal wound) that can be treated by the methods and composition described herein can be a wound that has failed to progress through the phases of healing in an orderly and timely fashion, does not improve after four weeks, and/or does not heal in eight weeks. Types of chronic wounds that can be treated by the compositions and methods described herein may include, but are not limited to venous ulcers, diabetic ulcers, pressure ulcers, non-healing surgical wounds, wounds related to metabolic diseases, wounds that repeatedly break down, and lesions and rashes associated with or caused by shingles, allergy (e.g., induced by irritants, such as certain chemical, plants, animals, etc.), and autoimmune diseases (e.g., psoriasis, dermatitis (e.g., atopic dermatitis (e.g., eczema), contact dermatitis, and seborrheic dermatitis), etc.). One or more chronic wounds (e.g., chronic dermal wounds) can be treated by the composition described herein, such as a composition containing a combination of bark resin extracts (e.g., aqueous bark resin extracts) of one or more plants of the genus Vismia (e.g., Vismia angusta) and one or more plants of the genus Calycophyllum (e.g., Calycophyllum spruceanum)).

In some embodiments, in the methods described herein, a chronic wound is treated by a composition that contains a combination of about 0.001-50% (w/v) bark resin extract (e.g., aqueous bark resin extract) from a plant of the genus Vismia (e.g., Vismia angusta) and about 0.001-50% (w/v) bark resin extract (e.g., aqueous bark resin extract) from a plant of the genus Calycophyllum (e.g., Calycophyllum spruceanum) in a ratio of 1:1. In other embodiments, in the methods described herein, a chronic wound is treated by a composition that contains a combination of about 0.001-50% (w/v) bark resin extract (e.g., aqueous bark resin extract) from a plant of the genus Vismia (e.g., Vismia angusta) and about 0.001-50% (w/v) bark resin extract (e.g., aqueous bark resin extract) from a plant of the genus Calycophyllum (e.g., Calycophyllum spruceanum) in a ratio of more than 1:1. Alternatively, in the methods described herein, a chronic wound is treated by a composition that contains a combination of about 0.001-50% (w/v) bark resin extract (e.g., aqueous bark resin extract) from a plant of the genus Vismia (e.g., Vismia angusta) and about 0.001-50% (w/v) bark resin extract (e.g., aqueous bark resin extract) from a plant of the genus Calycophyllum (e.g., Calycophyllum spruceanum) in a ratio of less than 1:1. Different ratios of bark resin extract (e.g., aqueous bark resin extract) from a plant of the genus Vismia and a plant of the genus Calycophyllum can differentially regulate and modulate the wound healing process. For example, in case of chronic wounds, increasing the ratio of bark resin extract (e.g., aqueous bark resin extract) from a plant of the genus Vismia to bark resin extract (e.g., aqueous bark resin extract) from a plant of the genus Calycophyllum may significantly reduce inflammation following extract-induced decrease in local TNF-α concentration, and increase in epidermal keratinocyte mitosis and wound closure. In particular, in the methods described herein, a chronic wound can be treated by a composition that contains a combination of about 0.001-50% (w/v) bark resin extract (e.g., aqueous bark resin extract) from a plant of the genus Vismia (e.g., Vismia angusta) and about 0.001-50% (w/v) bark resin extract (e.g., aqueous bark resin extract) from a plant of the genus Calycophyllum (e.g., Calycophyllum spruceanum) in a ratio of more than 1:1 (e.g., in a ratio of 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, or more (e.g., in a ratio of 10:1)). For example, in the methods described herein, chronic wounds (e.g., venous ulcers, diabetic ulcers, pressure ulcers, non-healing surgical wounds, wounds related to metabolic diseases, wound that repeatedly break down, and lesions and rashes associated with or caused by shingles, allergy (e.g., induced by irritants, such as certain chemical, plants, animals, etc.), and autoimmune diseases (e.g., psoriasis, dermatitis, etc.) can be treated by a composition that contains a combination of about 0.001-50% (w/v) bark resin extract (e.g., aqueous bark resin extract) from a plant of the genus Vismia (e.g., Vismia angusta) and about 0.001-50% (w/v) bark resin extract (e.g., aqueous bark resin extract) from a plant of the genus Calycophyllum (e.g., Calycophyllum spruceanum) in a ratio of 10:1.

### Acute Wounds

The compositions and methods described herein can be used for treating an acute wound. An acute wound (e.g., acute dermal wound) that can be treated by the methods and composition described herein can be an injury to the skin that occurs suddenly rather than over time, and heals at a predictable and expected rate according to the normal wound healing process. Types of acute wounds that can be treated by the compositions and methods described herein may include, but are not limited to trauma wounds (e.g., abrasions, lacerations, crush wounds, penetrations and punctures), surgical wounds (e.g., clean surgical wounds (e.g., made in an operating room or in a sterile procedure environment), contaminated surgical wounds (e.g., surgical wound contaminated with bacteria but not yet infected), and dirty surgical wounds (e.g., surgical wound with a bacterial infection)), and burn wounds. One or more acute wounds (e.g., acute dermal wounds) can be treated by the composition described herein, such as a composition containing a combination of bark resin extracts (e.g., aqueous bark resin extracts) of one or more plants of the genus Vismia (e.g., Vismia angusta) and one or more plants of the genus Calycophyllum (e.g., Calycophyllum spruceanum)).

In some embodiments, in the methods described herein, an acute wound is treated by a composition that contains a combination of about 0.001-50% (w/v) bark resin extract (e.g., aqueous bark resin extract) from a plant of the genus Vismia (e.g., Vismia angusta) and about 0.001-50% (w/v) bark resin extract (e.g., aqueous bark resin extract) from a plant of the genus Calycophyllum (e.g., Calycophyllum spruceanum) in a ratio of 1:1. In other embodiments, in the methods described herein, an acute wound is treated by a composition that contains a combination of about 0.001-50% (w/v) bark resin extract (e.g., aqueous bark resin extract) from a plant of the genus Vismia (e.g., Vismia angusta) and about 0.001-50% (w/v) bark resin extract (e.g., aqueous bark resin extract) from a plant of the genus Calycophyllum (e.g., Calycophyllum spruceanum) in a ratio of more than 1:1. Alternatively, in the methods described herein, an acute wound is treated by a composition that contains a combination of about 0.001-50% (w/v) bark resin extract (e.g., aqueous bark resin extract) from a plant of the genus Vismia (e.g., Vismia angusta) and about 0.001-50% (w/v) bark resin extract (e.g., aqueous bark resin extract) from a plant of the genus Calycophyllum (e.g., Calycophyllum spruceanum) in a ratio of less than 1:1. Different ratios of bark resin extract (e.g., aqueous bark resin extract) from a plant of the genus Vismia and a plant of the genus Calycophyllum can differentially regulate and modulate the wound healing process. For example, in case of acute wounds, epithelial scarring can be reduced by decreasing the ratio of bark resin extract (e.g., aqueous bark resin extract) from a plant of the genus Vismia to bark resin extract (e.g., aqueous bark resin extract) from a plant of the genus Calycophyllum, which results in moderate extract-induced reduction in inflammation, reduces or prevents bacterial and fungal colonization, decreases the rate of epidermal keratinocyte mitosis, and reduces fibrosis. In particular, in the methods described herein, an acute wound can be treated by a composition that contains a combination of about 0.001-50% (w/v) bark resin extract (e.g., aqueous bark resin extract) from a plant of the genus Vismia (e.g., Vismia angusta) and about 0.001-50% (w/v) bark resin extract (e.g., aqueous bark resin extract) from a plant of the genus Calycophyllum (e.g., Calycophyllum spruceanum) in a ratio of less than 1:1 (e.g., in a ratio of 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:21, 1:22, 1:23, 1:24, 1:25, 1:26, 1:27, 1:28, 1:29, 1:30, 1:31, 1:32, 1:33, 1:34, 1:35, 1:36, 1:37, 1:38, 1:39, 1:40, 1:41, 1:42, 1:43, 1:44, 1:45, 1:46, 1:47, 1:48, 1:49, 1:50, or less (e.g., in a ratio of 1:10)). For example, in the methods described herein, acute wounds (e.g., trauma wounds, surgical wounds, and burn wounds) can be treated by a composition that contains a combination of about 0.001-50% (w/v) bark resin extract (e.g., aqueous bark resin extract) from a plant of the genus Vismia (e.g., Vismia angusta) and about 0.001-50% (w/v) bark resin extract (e.g., aqueous bark resin extract) from a plant of the genus Calycophyllum (e.g., Calycophyllum spruceanum) in a ratio of 1:10. Treatment of wounds A composition described herein, such as a composition containing a combination of about 0.001-50% (w/v) bark resin extract (e.g., aqueous bark resin extract) from a plant of the genus Vismia (e.g., Vismia angusta) and about 0.001-50% (w/v) bark resin extract (e.g., aqueous bark resin extract) from a plant of the genus Calycophyllum (e.g., Calycophyllum spruceanum), can be used to treat a wound (e.g., a chronic wound or an acute wound) in a subject (e.g., a human subject or an animal model) in need thereof by administering to the subject an effective amount (e.g., a therapeutically effective amount) of the composition. In the methods described herein, the featured composition can be administered (e.g., topically) to the subject (e.g., at the wound site) in a dose (e.g., an effective amount) and for a time sufficient to treat the wound.

In some embodiments, a composition described herein, such as a composition containing a combination of about 0.001-50% (w/v) bark resin extract (e.g., aqueous bark resin extract) from a plant of the genus Vismia (e.g., Vismia angusta) and about 0.001-50% (w/v) bark resin extract (e.g., aqueous bark resin extract) from a plant of the genus Calycophyllum (e.g., Calycophyllum spruceanum), inhibits or reduces TNF-α release from cells, such as cells in a subject (e.g., a human subject or an animal model) or cells in a culture (e.g., a culture of peripheral blood mononuclear cells (PBMCs), such as a LPS-stimulated culture of PBMCs)), when the composition is administered to the subject (e.g., at the site of a wound (e.g., chronic or acute) in the subject) or to the cell culture in an amount (e.g., an effective amount) and for a time sufficient to inhibit or reduce TNF-α release. The featured composition can inhibit or reduce TNF-α release from cells by 5% or more (e.g., between 5-20%, between 5-50%, between 10-50%, between 10-80%, between 20-80%, or between 20-100% (e.g., 5%, 10%, 15%, 20%, 25%, 30%, 40%, 50%, 60%, 70%, 80%, 90%, 95%, 99%, or more)), compared to before the administration of the composition.

In some embodiments, a composition described herein, such as a composition containing a combination of about 0.001-50% (w/v) bark resin extract (e.g., aqueous bark resin extract) from a plant of the genus Vismia (e.g., Vismia angusta) and about 0.001-50% (w/v) bark resin extract (e.g., aqueous bark resin extract) from a plant of the genus Calycophyllum (e.g., Calycophyllum spruceanum), induces or increases migration of epidermal keratinocytes, such as epidermal keratinocytes in a subject (e.g., a human subject or an animal model) or epidermal keratinocytes in a culture, when the composition is administered to the subject (e.g., at the site of a wound (e.g., chronic or acute) in the subject) or to the cell culture in an amount (e.g., an effective amount) and for a time sufficient to induce or increase migration of epidermal keratinocytes. The featured composition can induce or increase migration of epidermal keratinocytes by 5% or more (e.g., between 5-20%, between 5-50%, between 10-50%, between 10-80%, between 20-80%, or between 20-100% (e.g., 5%, 10%, 15%, 20%, 25%, 30%, 40%, 50%, 60%, 70%, 80%, 90%, 95%, 99%, or more)), compared to before the administration of the composition.

In some embodiments, a composition described herein, such as a composition containing a combination of about 0.001-50% (w/v) bark resin extract (e.g., aqueous bark resin extract) from a plant of the genus Vismia (e.g., Vismia angusta) and about 0.001-50% (w/v) bark resin extract (e.g., aqueous bark resin extract) from a plant of the genus Calycophyllum (e.g., Calycophyllum spruceanum), induces or increases contracture of wound (e.g., chronic wound or acute wound) in a subject (e.g., a human subject or an animal model), when the composition is administered to the subject (e.g., at the site of the wound in the subject) in an amount (e.g., an effective amount) and for a time sufficient to induce or increase wound contracture. The featured composition can induce or increase wound contracture by 5% or more (e.g., between 5-20%, between 5-50%, between 10-50%, between 10-80%, between 20-80%, or between 20-100% (e.g., 5%, 10%, 15%, 20%, 25%, 30%, 40%, 50%, 60%, 70%, 80%, 90%, 95%, 99%, or more)), compared to before the administration of the composition.

In some embodiments, a composition described herein, such as a composition containing a combination of about 0.001-50% (w/v) bark resin extract (e.g., aqueous bark resin extract) from a plant of the genus Vismia (e.g., Vismia angusta) and about 0.001-50% (w/v) bark resin extract (e.g., aqueous bark resin extract) from a plant of the genus Calycophyllum (e.g., Calycophyllum spruceanum), induces or increases closure of wound (e.g., chronic wound or acute wound) in a subject (e.g., a human subject or an animal model), when the composition is administered to the subject (e.g., at the site of the wound in the subject) in an amount (e.g., an effective amount) and for a time sufficient to induce or increase wound closure. The featured composition can induce or increase wound closure by 5% or more (e.g., between 5-20%, between 5-50%, between 10-50%, between 10-80%, between 20-80%, or between 20-100% (e.g., 5%, 10%, 15%, 20%, 25%, 30%, 40%, 50%, 60%, 70%, 80%, 90%, 95%, 99%, or more)), compared to before administration of the composition. In some embodiments, a wound treated with the featured composition can close at least 5% (e.g., between 5-20%, between 5-50%, between 10-50%, between 10-80%, between 20-80%, or between 20-100% (e.g., 5%, 10%, 15%, 20%, 25%, 30%, 40%, 50%, 60%, 70%, 80%, 90%, 95%, 99%, or more)) faster, compared to before the administration of the composition, or compared to a wound that is not treated with the composition.

In some embodiments, a composition described herein, such as a composition containing a combination of about 0.001-50% (w/v) bark resin extract (e.g., aqueous bark resin extract) from a plant of the genus Vismia (e.g., Vismia angusta) and about 0.001-50% (w/v) bark resin extract (e.g., aqueous bark resin extract) from a plant of the genus Calycophyllum (e.g., Calycophyllum spruceanum), induces healing of wound (e.g., chronic wound or acute wound) in a subject (e.g., a human subject or an animal model), when the composition is administered to the subject (e.g., at the site of the wound in the subject) in an amount (e.g., an effective amount) and for a time sufficient to induce wound healing. The featured composition can induce wound healing by 5% or more (e.g., between 5-20%, between 5-50%, between 10-50%, between 10-80%, between 20-80%, or between 20-100% (e.g., 5%, 10%, 15%, 20%, 25%, 30%, 40%, 50%, 60%, 70%, 80%, 90%, 95%, 99%, or more)), compared to before the administration of the composition. In some embodiments, a wound treated with the featured composition can heal at least 5% (e.g., between 5-20%, between 5-50%, between 10-50%, between 10-80%, between 20-80%, or between 20-100% (e.g., 5%, 10%, 15%, 20%, 25%, 30%, 40%, 50%, 60%, 70%, 80%, 90%, 95%, 99%, or more)) faster, compared to before the administration of the composition, or compared to a wound that is not treated with the composition. In some embodiments, a wound treated with the featured composition can heal in about 8 weeks or less (e.g., about 8 weeks, about 7 weeks, about 6 weeks, about 5 weeks, about 4 weeks, about 3 weeks, about 2 weeks, about 13 days, about 12 days, about 11 days, about 10 days, about 9 days, about 8 days, about 7 days, about 6 days, about 5 days, about 4 days, about 3 days, about 2 days, about 1 day, about 12 hours, or less) following the administration of the composition.

In some embodiments, a composition described herein, such as a composition containing a combination of about 0.001-50% (w/v) bark resin extract (e.g., aqueous bark resin extract) from a plant of the genus Vismia (e.g., Vismia angusta) and about 0.001-50% (w/v) bark resin extract (e.g., aqueous bark resin extract) from a plant of the genus Calycophyllum (e.g., Calycophyllum spruceanum), inhibits or reduces inflammation (e.g., inhibits or reduces release of inflammatory cytokines (e.g., TNF-α, IFNγ, IL-1, IL-6, IL-1p, IL-12, IL-18, etc.) from cells) in a subject (e.g., a human subject or an animal model) or in a cell culture (e.g., a culture of PBMCs, such as a LPS-stimulated culture of PBMCs), when the composition is administered to the subject (e.g., at the site of a wound (e.g., chronic or acute) in the subject) or to the cell culture in an amount (e.g., an effective amount) and for a time sufficient to inhibit or reduce inflammation (e.g., inhibit or reduce release of inflammatory cytokines). The featured composition can inhibit or reduce inflammation by 5% or more (e.g., between 5-20%, between 5-50%, between 10-50%, between 10-80%, between 20-80%, or between 20-100% (e.g., 5%, 10%, 15%, 20%, 25%, 30%, 40%, 50%, 60%, 70%, 80%, 90%, 95%, 99%, or more)), compared to before administration of the composition.

### Formulations and Carriers

In order to be administered to a subject (e.g., a human subject or an animal model), a composition described herein, such as a composition containing a combination of about 0.001-50% (w/v) bark resin extract (e.g., aqueous bark resin extract) from a plant of the genus Vismia (e.g., Vismia angusta) and about 0.001-50% (w/v) bark resin extract (e.g., aqueous bark resin extract) from a plant of the genus Calycophyllum (e.g., Calycophyllum spruceanum), can be formulated as a pharmaceutical composition. Pharmaceutical compositions or formulations contemplated herein include combination of bark resin extracts (e.g., aqueous bark resin extracts) from Vismia spp. and Calycophyllum spp. with a pharmaceutically acceptable carrier, adjuvant or vehicle. A pharmaceutically acceptable carrier or excipient refers to a carrier (e.g., carrier, media, diluent, solvent, vehicle, etc.) which does not significantly interfere with the biological activity or effectiveness of the active ingredient(s) of a pharmaceutical composition (e.g., active ingredient(s) of bark resin extracts from Vismia spp. and Calycophyllum spp.) and which is not excessively toxic to the host at the concentrations at which it is used or administered. Pharmaceutical compositions or formulations contemplated herein may include carriers (e.g., diluents, excipients and auxiliaries) that facilitate processing of the active components (e.g., active ingredient(s) of bark resin extracts from Vismia spp. and Calycophyllum spp.) into a pharmaceutically acceptable formulation. Carriers employed may be either solid or liquid. Exemplary solid carriers are lactose, sucrose, talc, gelatin, agar, pectin, acacia, magnesium stearate, stearic acid, and the like. Exemplary liquid carriers are syrup, peanut oil, olive oil, water, and the like. Similarly, the inventive compositions (e.g., pharmaceutical compositions containing bark resin extracts from Vismia spp. and Calycophyllum spp.) may include time-delay or time-release material known in the art, such as glyceryl monostearate or glyceryl distearate alone or with a wax, ethylcellulose, hydroxypropylmethylcellulose, methylmethacrylate, or the like. Further additives or excipients may be added to achieve the desired formulation properties. For example, a bioavailability enhancer, such: as LABRASOL®, GELUCIRE®, or the like, or formulators, such as CHIC (carboxy-methylcellulose), PG (propyleneglycol), or PEG (polyethyleneglycol), may be added. GELUCIRE®, a semi-solid vehicle that protects active ingredients from light, moisture and oxidation, may be added, e.g., when preparing a capsule formulation. Other pharmaceutically acceptable ingredients can be present in the composition as well. Suitable substances and their use for the formulation of pharmaceutically active compounds are well-known in the art (see, for example, Remington: The Science and Practice of Pharmacy 22^{th} edition (2012), for additional discussion of pharmaceutically acceptable substances and methods of preparing pharmaceutical compositions of various types).

If a solid carrier is used, the preparation can be tableted, placed in a hard gelatin capsule in powder or pellet form, or formed into a troche or lozenge. The amount of solid carrier may vary, but generally will be from about 25 mg to about 1 g. If a liquid carrier is used, the preparation may be in the form of ointment, lotion, gel, cream, salve, liniment, paste, tonic, unguent, spray, soap, shampoo, lip balm, syrup, emulsion, soft gelatin capsule, sterile injectable solution or suspension in an ampoule or vial or non-aqueous liquid suspension. Further, the pharmaceutical composition may be incorporated into a skin patch for delivery of the drug directly onto the skin. The inventive compositions are prepared in unit-dosage form appropriate for the mode of administration, e.g., parenteral (e.g., topical) or oral administration.

To obtain a stable water-soluble dose form, the active components of the present invention (bark resin extracts from Vismia spp. and Calycophyllum spp.) may be dissolved in water, or an aqueous solution of an organic or inorganic acid, such as 0.3 M solution of succinic acid or citric acid. The active components may also be dissolved in a suitable co-solvent or combinations of co-solvents. Examples of suitable co-solvents include alcohol, propylene glycol, polyethylene glycol 300, polysorbate 80, glycerin, and the like in concentrations ranging from 0-60% of the total volume. For example, an active component of the present invention can be dissolved in DMSO and diluted with water. The composition may also be in the form of a solution of a salt form of the active ingredient in an appropriate aqueous vehicle such as water or isotonic saline or dextrose solution.

Pharmaceutically acceptable carriers, adjuvants and vehicles that may be used in the pharmaceutical compositions or formulations of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethyleneglycol, sodium carboxymethylcellulose, polyacrylates, waxes, polyethylene-polyoxypropylene-block polymers, polyethylene glycol, liposomes and wool fat. In some embodiments, the vehicle used for preparing the pharmaceutical composition or formulation featured herein is water. In particular, the pharmaceutical composition or formulation featured herein may contain at least 40% (e.g., at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, or more) water. For example, the pharmaceutical composition or formulation featured herein, such as a composition containing a combination of bark resin extracts (e.g., aqueous bark resin extracts) from Vismia spp. and Calycophyllum spp. with a pharmaceutically acceptable carrier, adjuvant or vehicle, may contain at least 50% water.

The composition of this invention may be modified by appending appropriate functionalities to enhance selective biological properties. Such modifications are known in the art and include those that increase biological penetration into a given biological system (e.g., blood, lymphatic system, central nervous system), increase oral bioavailability, increase solubility to allow administration by injection, alter metabolism, or alter rate of excretion (Pharmacokinetic Optimization in Drug Research, Testa, B. et al., 2001, Wiley-VCH, VCHA).

A pharmaceutical composition is typically formulated to be compatible with its intended route of administration. The pharmaceutical compositions of this invention may be administered parenterally (e.g., topically), orally, by inhalation spray, rectally, nasally, buccally, vaginally, or via an implanted reservoir, and are preferably administered topically. The pharmaceutical compositions of this invention may contain any conventional non-toxic pharmaceutically acceptable carriers, adjuvants or vehicles. The term “parenteral” or “parenterally” as used herein includes topical administration, injections (e.g., sub-cutaneous, intra-cutaneous, intra-venous, intra-muscular, intra-articular, intra-synovial, intra-sternal, intra-thecal, intra-lesional and intracranial injection) or infusion techniques.

For topical application, a pharmaceutical composition may be formulated in a suitable ointment, lotion, gel, cream, salve, liniment, paste, tonic, unguent, spray, soap, shampoo, or lip balm containing the active components (e.g., bark resin extracts (e.g., aqueous bark resin extracts) from Vismia spp. and Calycophyllum spp.) suspended or dissolved in one or more pharmaceutically acceptable carriers suitable for use in such compositions.

For administration by injection, pharmaceutical compositions of this invention can be formulated in the form of a sterile injectable preparation, for example, as a sterile injectable aqueous or oleaginous suspension using a sterile solution or any pharmaceutically acceptable liquid as a vehicle. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, for example, as a solution in 1,3-butanediol. Among the acceptable vehicles and solvents that may be employed are mannitol, water, Ringer's solution and isotonic sodium chloride solutions. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. Pharmaceutically acceptable vehicles also include, but are not limited to, cell culture media (e.g., Dulbecco's Modified Eagle Medium (DMEM), α-Modified Eagles Medium (α-MEM), F-12 medium). Formulation methods are known in the art, see e.g., Banga (ed.) Therapeutic Peptides and Proteins: Formulation, Processing and Delivery Systems (3rd ed.) Taylor & Francis Group, CRC Press (2015).

Pharmaceutical compositions of the invention may also be administered in the form of suppositories for rectal administration. These compositions can be prepared by mixing the composition described herein (e.g., composition containing bark resin extracts (e.g., aqueous bark resin extracts) from Vismia spp. and Calycophyllum spp.) with a suitable non-irritating excipient that is solid at room temperature but liquid at the rectal temperature and therefore will melt in the rectum to release the active components. Such materials include, but are not limited to, cocoa butter, beeswax, and polyethylene glycols.

For administration by inhalation, pharmaceutical compositions of this invention may be formulated in the form of an aerosol spray from a pressured container or dispenser, which contains a suitable propellant, e.g., a gas such as carbon dioxide, a fluorocarbon, or a nebulizer. Liquid or dry aerosol (e.g., dry powders, large porous particles, etc.) can also be used. The pharmaceutical compositions of this invention may be administered by nasal aerosol or inhalation. Such compositions are prepared according to techniques well-known in the art of pharmaceutical formulation and may be prepared as solutions in saline employing benzyl alcohol or other suitable preservatives, absorption promoters to enhance bioavailability, fluorocarbons, and/or other solubilizing or dispersing agents known in the art.

For oral administration, agents can be formulated by combining the active compounds with pharmaceutically acceptable carriers well known in the art. Such carriers enable the compounds of the invention to be formulated as a powder, tablet, pill, capsule, lozenge, liquid, gel, syrup, slurry, suspension, and the like. It is recognized that some pharmaceutical compositions, if administered orally, must be protected from digestion. This is typically accomplished either by complexing the protein with a composition to render it resistant to acidic and enzymatic hydrolysis or by packaging the protein in an appropriately resistant carrier such as a liposome. Suitable excipients for oral dosage forms include, for example, fillers such as sugars, including lactose, sucrose, mannitol, or sorbitol; cellulose preparations such as, for example, starch, gelatin, gum tragacanth, methyl cellulose, hydroxypropylmethyl cellulose, sodium carboxymethylcellulose, and/or polyvinylpyrrolidone (PVP). Disintegrating agents may be added, for example, such as the cross linked polyvinyl pyrrolidone, agar, or alginic acid or a salt thereof such as sodium alginate. Lubricating agents, such as magnesium stearate, are also typically added. When aqueous suspensions are administered orally, the active ingredient is combined with emulsifying and suspending agents. If desired, certain sweetening, flavoring, and/or coloring agents may be added. Optionally the oral formulations may also be formulated in saline or buffers for neutralizing internal acid conditions or may be administered without any carriers.

Pharmaceutical compositions may also be prepared in microcapsules, such as hydroxylmethylcellulose or gelatin-microcapsule and poly-(methylmethacrylate) microcapsule. Pharmaceutical compositions containing a combination of bark resin extracts (e.g., aqueous bark resin extracts) from Vismia spp. and Calycophyllum spp. may also be prepared in other drug delivery systems such as liposomes, albumin microspheres, microemulsions, nano-particles, and nanocapsules. Such techniques are described in the art. The pharmaceutical compositions to be used for in vivo administration must be sterile. This is readily accomplished by filtration through sterile filtration membranes.

### Combination Therapy

The composition described herein (e.g., a pharmaceutical composition described hereinabove, such as a composition containing a combination of bark resin extracts (e.g., aqueous bark resin extracts) from Vismia spp. and Calycophyllum spp. and a pharmaceutically acceptable carrier, adjuvant or vehicle) can be used as a monotherapy (e.g., the composition can be administered alone for treating wounds) or a combination therapy (e.g., the composition can be administered with one or more additional agents for treating wounds). In some embodiments, the composition described herein can be administered (e.g., topically) to a subject (e.g., a human subject or an animal model) as a combination therapy, e.g., along with one or more additional agents. Such additional agents may include, but are not limited to, one or more antibiotic agents, antiseptic agents, antifungal agents, antiviral agents, analgesic agents, UV-absorbing agents, and moisturizing agents.

In some embodiments, the additional agents (e.g., antibiotic agents, antiseptic agents, antifungal agents, antiviral agents, analgesic agents, UV-absorbing agents, and/or moisturizing agents) can be included in the pharmaceutical composition or formulation contemplated herein. For example, a combination therapy may include administering (e.g., topically) to a subject (e.g., a human subject or an animal model) a pharmaceutical composition or formulation that contains a combination of bark resin extracts (e.g., aqueous bark resin extracts) from Vismia spp. and Calycophyllum spp., one or more pharmaceutically acceptable carriers, adjuvants or vehicles, and one or more additional agents (e.g., antibiotic agents, antiseptic agents, antifungal agents, antiviral agents, analgesic agents, UV-absorbing agents, and/or moisturizing agents).

In other embodiments, the additional agents (e.g., antibiotic agents, antiseptic agents, antifungal agents, antiviral agents, analgesic agents, UV-absorbing agents, and/or moisturizing agents) can be administered (e.g., parenterally (e.g., topically) or orally) as a separate composition (e.g., a separate pharmaceutical composition) prior to, concurrent with, or subsequent to administration (e.g., topical administration) of the composition (e.g., pharmaceutical composition or formulation) described herein. For example, a combination therapy may include administering (e.g., parenterally (e.g., topically) or orally) to a subject (e.g., a human subject or an animal model) one or more additional agents (e.g., antibiotic agents, antiseptic agents, antifungal agents, antiviral agents, analgesic agents, UV-absorbing agents, and/or moisturizing agents) prior to, concurrent with, or subsequent to administration (e.g., topical administration) of a pharmaceutical composition or formulation that contains a combination of bark resin extracts (e.g., aqueous bark resin extracts) from Vismia spp. and Calycophyllum spp., and one or more pharmaceutically acceptable carriers, adjuvants or vehicles.

Antibiotic agents that can be used in combination therapy with a composition (e.g., pharmaceutical composition) described herein (e.g., antibiotic agents that can be included in a pharmaceutical composition described herein or administered prior to, concurrent with, or subsequent to administration of a pharmaceutical composition described herein) may be selected from: mupirocin, bacitracin, polymyxin B, and neomycin.

Antiseptic agents that can be used in combination therapy with a composition (e.g., pharmaceutical composition) described herein (e.g., antiseptic agents that can be included in a pharmaceutical composition described herein or administered prior to, concurrent with, or subsequent to administration of a pharmaceutical composition described herein) may be selected from: sodium hypochlorite, chlorhexidine, hexachlorophene, povidone iodine, ethanol, benzethonium chloride, triclosan, and benzalkonium chloride.

Antifungal agents that can be used in combination therapy with a composition (e.g., pharmaceutical composition) described herein (e.g., antifungal agents that can be included in a pharmaceutical composition described herein or administered prior to, concurrent with, or subsequent to administration of a pharmaceutical composition described herein) may be selected from: Whitfield ointment (3% salicylic acid, 6% benzoic acid in petrolatum), Undecylenic alkanolamide, Ciclopirox olamine, Polyenes (e.g., Nystatin), Imidazoles (e.g., Bifonazole, Clotrimazole, Econazole, Efinaconazole, Ketoconazole, Luliconazole, Miconazole, Sulconazole, Tioconazole), Allylamine (e.g., Terbinafine), Thiocarbamates (e.g., Tolciclate, Tolnaftate), and Benzoxaborole (e.g., Tavaborole).

Antiviral agents that can be used in combination therapy with a composition (e.g., pharmaceutical composition) described herein (e.g., antiviral agents that can be included in a pharmaceutical composition described herein or administered prior to, concurrent with, or subsequent to administration of a pharmaceutical composition described herein) may be selected from: acyclovir, and penciclovir.

Analgesic agents that can be used in combination therapy with a composition (e.g., pharmaceutical composition) described herein (e.g., analgesic agents that can be included in a pharmaceutical composition described herein or administered prior to, concurrent with, or subsequent to administration of a pharmaceutical composition described herein) may be selected from: Benzocaine (Anbesol, Cepacol®, others), Benzocaine/Butamben/Tetracaine (Cetacaine®), Benzyl Alcohol (Ulesfia; Zilactin), Capsaicin (various), Dibucaine (Nupercainal), Diclofenac (Flector®, Pennsaid®, Voltaren®, others), Dyclonine (Dyclocaine®, Sucrets®), Ethyl Chloride, Hexylresorcinol (Sucrets®), Lidocaine (Akten, AneCream, Lidoderm, Xylocaine, others), Lidocaine/Prilocaine (EMLA®, Oraqix®), Lidocaine/Tetracaine (Synera®), Methyl alicylate/Menthol (BenGay®; Icy Hot®, others), Pramoxine (Itch-X, Proctofoam, others), Proparacaine (Flucaine), Tetracaine (Altacaine®, Pontocaine®, others), and Trolamine (Arthricream, Myoflex, others).

UV-absorbing agents that can be used in combination therapy with a composition (e.g., pharmaceutical composition) described herein (e.g., UV-absorbing agents that can be included in a pharmaceutical composition described herein or administered prior to, concurrent with, or subsequent to administration of a pharmaceutical composition described herein) may be selected from: Phenyl ester of salicylate (Component: phenyl o-hydroxybenzoate); UV-absorber UV-P (Component: o-nitroaniline, p-cresol reaction products); UV-absorbing agent UV-O (Component: 2,4-dihydroxybenzophenone); Ultraviolet absorber UV-9 (Component: 2-hydroxy-4-methoxybenzophenone); UV-absorbing agent UV-531 (Component: 2-hydroxy-4-n-octoxybenzophenone); UV absorber UVP-327 (Component: 2-(2′-hydroxy-3′, 5′-di-tert-phenyl)-5-chlorobenzotriazole); UV absorbers (Component: resorcinol monobenzoate); Light stabilizer AM-101 (Composition: 2,2′-thiobis (4-t-octylphenoxy) nickel); Light stabilizer GW-540 (Component: tris (1,2,2,6,6-pentamethylpiperidyl) phosphite); Light stabilizer 744 (Component: 4-benzoyloxy-2,2,6,6-tetramethylpiperidine); and Light stabilizer HPT (Ingredients: hexamethylphosphoric triamide).

Moisturizing agents that can be used in combination therapy with a composition (e.g., pharmaceutical composition) described herein (e.g., moisturizing agents that can be included in a pharmaceutical composition described herein or administered prior to, concurrent with, or subsequent to administration of a pharmaceutical composition described herein) may be selected from: humectants (e.g., glycerol, sorbitol, glycerin, urea, lactic acid, and hyaluronic acid) and emollients (e.g., lanolin, paraffin, ceramides, and silicones).

### Method of Treatment

Methods of treatment, their dosage levels and requirements featured herein may be selected by those of ordinary skill in the art from available methods and techniques.

It will be appreciated that the actual dosages of the composition (e.g., pharmaceutical composition) of this invention (e.g., pharmaceutical composition or formulation containing a combination of bark resin extracts (e.g., aqueous bark resin extracts) from Vismia spp. and Calycophyllum spp., and one or more pharmaceutically acceptable carriers, adjuvants or vehicles) as a monotherapy or a combination therapy will vary according to the particular composition formulated, the mode of administration, the particular wound being treated, and the particular subject being treated. Those skilled in the art using conventional dosage-determination tests in view of the experimental data may ascertain optimal dosages for a given set of conditions. For topical administration, an exemplary dose will be about 0.001-50% (w/v) of the bark resin extract from Vismia spp. and about 0.001-50% (w/v) of the bark resin extract from Calycophyllum spp., as described in the foregoing sections. The course of treatment can be repeated (e.g., 1-12 times or more (e.g., 1 time, 2 times, 3 times, 4 times, 5 times, 6 times, 7 times, 8 times, 9 times, 10 times, 11 times, 12 times, or more)) at appropriate intervals, e.g., every 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 1 week, 2 weeks, 3 weeks, 4 weeks, 5 weeks, 6 weeks, 7 weeks, 8 weeks, 3 months, 4 months, 5 months, 6 months, 7 months, 8 months, 9 months, 10 months, 11 months, or 1 year. The composition described herein may be administered to a subject in need thereof, for example, one or more times (e.g., 1-12 times or more) hourly, daily, weekly, biweekly, monthly, bimonthly, quarterly, biannually, annually, or as medically necessary. Dosages may be provided in either a single or multiple dosage regimens. The timing between administrations may decrease as the medical condition improves or increase as the health of the patient declines. The treatment may be continued for 5 years or less (e.g., 5 years, 4 years, 3 years, 2 years, 1 year, 6 months, 5 months, 4 months, 3 months, 8 weeks, 7 weeks, 6 weeks, 5 weeks, 4 weeks, 3 weeks, 15 days, 14 days, 13 days, 12 days, 11 days, 10 days, 9 days, 8 days, 7 days, 6 days, 5 days, 4 days, 3 days, 2 days, or less). For example, a wound (e.g., chronic or acute) in a subject may be treated by administering (e.g., topically) to the subject a composition (e.g., pharmaceutical composition) containing a combination of 0.001-50% (w/v) of the bark resin extract from Vismia spp. and about 0.001-50% (w/v) of the bark resin extract from Calycophyllum spp. as a monotherapy (e.g., the composition alone) or a combination therapy (e.g., the composition with one or more additional agents (e.g., antibiotic agents, antiseptic agents, antifungal agents, antiviral agents, analgesic agents, UV-absorbing agents, and/or moisturizing agents)) for 8 weeks or less (e.g., 8 weeks, 7 weeks, 6 weeks, 5 weeks, 4 weeks, 3 weeks, 15 days, 14 days, 13 days, 12 days, 11 days, 10 days, 9 days, 8 days, 7 days, 6 days, 5 days, 4 days, 3 days, 2 days, or less).

Upon improvement of a patient's condition, a maintenance dose of a composition of this invention may be administered if necessary. Subsequently, the dosage or frequency of administration, or both, may be reduced, as a function of the symptoms, to a level at which the improved condition is retained. When the symptoms have been reduced or alleviated to the desired level, treatment should cease, at least in principle. Patients may, however, require intermittent treatment on a long-term basis, upon any recurrence of wound.

As the skilled artisan will appreciate, lower or higher doses than those recited above may be required. Specific dosage and treatment regimen for any particular patient will depend upon a variety of factors, including the activity of the specific composition used, the age, body weight, general health status, sex, diet, time of administration, rate of excretion, the severity of the wound, the patient's disposition to the wound and the judgment of the treating physician.

With respect to the composition of the present invention, the particular pharmaceutical formulation, the dosage, and the number of doses given per day to a mammal requiring such treatment are all choices within the knowledge of one of ordinary skill in the art and can be determined without undue experimentation.

### EXAMPLES

The following examples are put forth to provide those of ordinary skill in the art with a description of how the compositions described herein may be prepared and used, and how the methods featured herein may be evaluated. The examples are intended to be purely exemplary of the invention and are not intended to limit the scope of the claims.

### Example 1. Materials and Methods

Bark resin from Vismia angusta and Calycophyllum spruceanum were procured from the Loreto region of Peru. Pieces of bark from each tree, approximately 15 cm×30 cm in size, were removed and vacuum sealed on site in order to protect the bark resins from oxidation. During the extraction process, the bark was removed from the subject tree and then immediately sealed into a protective casing, preferably, ‘FoodSaver®’ brand one-gallon, or one-quart size, vacuum zipper preservation bags. Using a portable ‘FoodSaver®’ air pump, air was removed from the sample ‘FoodSaver®’ bag and the sample was stored in a large backpack to prevent exposure to light and heat. The entire process from extraction of the bark to storage in the sealed pack was completed in about thirty to sixty seconds. The secured sample was stored in an air-conditioned environment and periodically checked until arrival in Rhode Island. This process allowed maintenance of integrity of the samples for a significant period of time ranging from 1-10 days without decay or degradation prior to storage in −20° Celsius freezer.

Upon arrival at the Rhode Island based laboratory, individual samples of bark were cut into 1 cm squared pieces and ground into fine pulp. 100 g of each type of bark pulp were vacuum sealed separately and stored in a −20° Celsius frosted freezer until use.

For preparation of bark resin extracts, 100 g of vacuum sealed samples were removed from the low temperature freezer and thawed in a 4° Celsius refrigerator overnight. Following thawing, 100 g of each pulp were wrapped separately in cheese cloth and secured on each end in order to contain the pulp. Individually packaged pulp was then inserted and pressed 3 times through a commercially available manual sugar cane press juicer, Juice Machine Extractor Mill 50 kg/h. 40 ml+/−3 ml of extracted Vismia bark resin and 37 ml+/−1 ml of extracted Calycophyllum bark resin were consistently collected from each 100 g of bark pulp. Extracted liquid resins were then combined into formulations, as stated in the embodiments.

### Example 2. Combination of Bark Resin Extracts from Vismia angusta and Calycophyllum Spruceanum Inhibits TNF-α Release

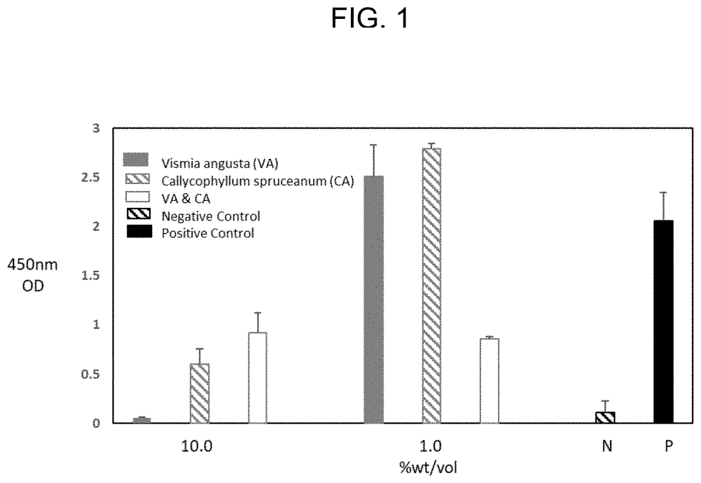

Human peripheral blood mononuclear cells (PBMCs) were incubated for 12 hours in: medium alone (negative control, N); medium+LPS (positive control, P); medium+LPS+Vismia angusta bark resin extract (VA); medium+LPS+Calycophyllum spruceanum bark resin extract (CA); or medium+LPS+Vismia angusta bark resin extract+Calycophyllum spruceanum bark resin extract (VACA) under standard tissue culture conditions for 12 hours. Cell supernatants were then tested by ELISA for the presence of PBMC-secreted TNF-α. The data shows significant inhibition of TNF-α release from PBMCs incubated with the combination of Vismia angusta and Calycophyllum spruceanum bark resin extracts (VACA) as compared to the positive control (P). The data also show improved properties of the combination of Vismia angusta and Calycophyllum spruceanum bark resin extracts (VACA), when used at a 1% (w/v) concentration, over the individual bark resin extracts (VA or CA) (FIG. 1). This indicates that a composition described in the present disclosure containing a combination of bark resin extracts from Vismia spp. and Calycophyllum spp. (as represented by VACA in FIG. 1) is more effective in reducing TNF-α release and inflammation compared to the individual bark resin extracts (as represented by VA or CA in FIG. 1).

### Example 3. A Combination of Vismia angusta and Calycophyllum spruceanum Bark Resin Extracts Promotes Wound Healing in an In Vitro Wound Healing Assay

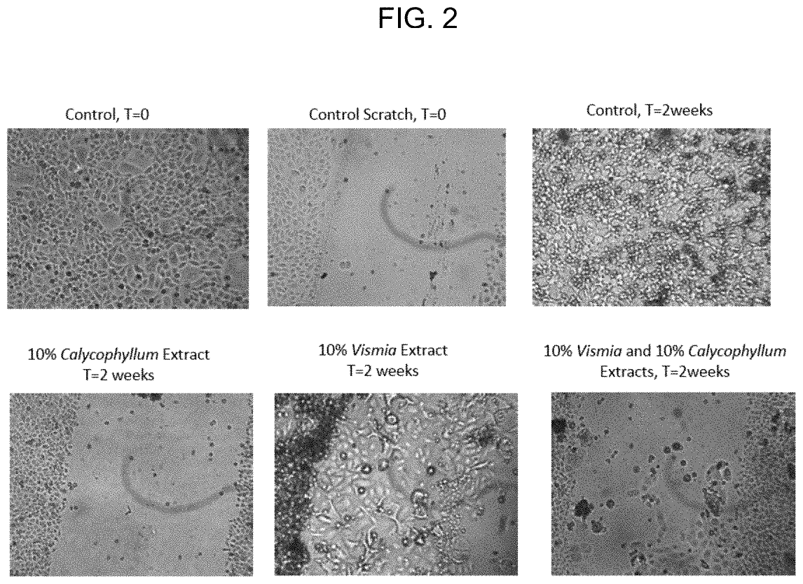

Human epidermal keratinocytes were cultured to confluence. A standard in vitro scratch assay was performed in which a 0.9 mm diameter wound was made through the midline of each confluent cell culture well. Cell culture wells were incubated for up to 2 weeks with medium alone (Control), Vismia angusta alone, Calycophyllum spruceanum alone, or a combination of Vismia angusta and Calycophyllum spruceanum bark resin extracts (10.0%, 1.0%, or 0.01% (w/v) concentration). Cell culture wells containing all concentrations of Vismia and Calycophyllum bark resin extracts showed controlled migration and wound closure of human epidermal keratinocytes compared to the control wells. Representative data using 10% (w/v) Vismia angusta bark resin extract, 10% (w/v) Calycophyllum spruceanum bark resin extract, and a combination of 10% (w/v) Vismia angusta and 10% (w/v) Calycophyllum spruceanum bark resin extracts, are shown in FIG. 2. This indicates that a composition described in the present disclosure containing a combination of bark resin extracts from Vismia spp. and Calycophyllum spp. (as represented by the bottom right micrograph in FIG. 2) can effectively modulate keratinocyte migration and wound healing.

### Example 4. A Combination of Vismia angusta and Calycophyllum spruceanum Bark Resin Extracts Promotes Healing of Chronic Wound in Human

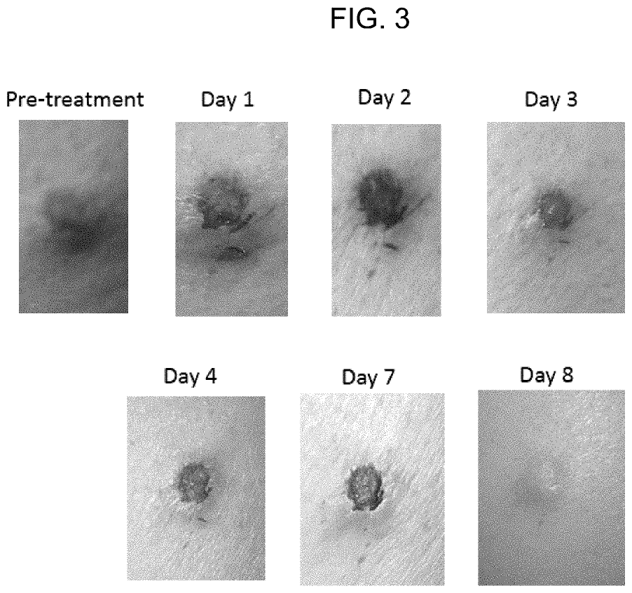

A composition containing 10% (w/v) Vismia angusta bark resin extract and 10% (w/v) Calycophyllum spruceanum bark resin extract was applied topically to a human subject on a dermal ulceration caused by an insect bite. FIG. 3 shows progression of wound healing over a period of 1-8 days following topical administration of the composition. This indicates the effectiveness of a composition described in the present disclosure (e.g., containing a combination of bark resin extracts from Vismia spp. and Calycophyllum spp.) in inducing wound healing.

### Example 5. A Combination of Vismia angusta and Calycophyllum spruceanum Bark Resin Extracts Promotes Healing of Shingles Rash in Human

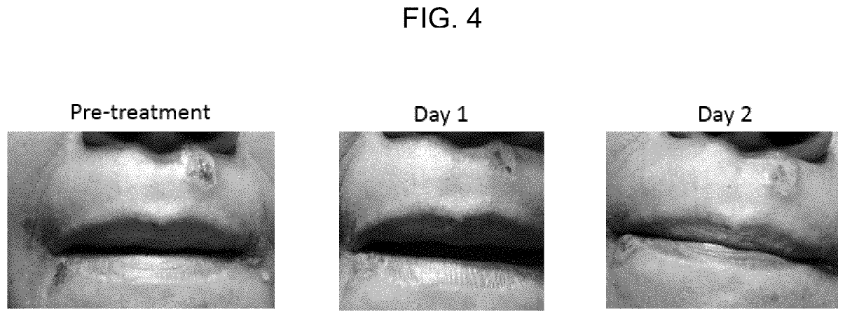

A composition containing 10% (w/v) Vismia angusta bark resin extract and 10% (w/v) Calycophyllum spruceanum bark resin extract was applied topically to a human subject on a dermal ulceration associated with diagnosed shingles infection. FIG. 4 shows progression of wound healing over a period of 2 days following topical administration of the composition. This indicates the effectiveness of a composition described in the present disclosure (e.g., containing a combination of bark resin extracts from Vismia spp. and Calycophyllum spp.) in inducing wound healing.

### OTHER EMBODIMENTS

While the invention has been described in connection with specific embodiments thereof, it will be understood that it is capable of further modifications and this application is intended to cover any variations, uses, or adaptations of the invention following, in general, the principles of the invention and including such departures from the present disclosure come within known or customary practice within the art to which the invention pertains and may be applied to the essential features hereinbefore set forth.

All publications, patents, and patent applications are herein incorporated by reference in their entirety to the same extent as if each individual publication, patent or patent application was specifically and individually indicated to be incorporated by reference in its entirety.

1. A pharmaceutical composition comprising about 0.001-50% (w/v) bark resin extract from a plant of genus Vismia and about 0.001-50% w/v bark resin extract from a plant of genus Calycophyllum.

2. The pharmaceutical composition of claim 1, wherein the pharmaceutical composition comprises about 0.01-50% (w/v) bark resin extract from the plant of genus Vismia and about 0.001-50% w/v bark resin extract from the plant of genus Calycophyllum.

3. The pharmaceutical composition of claim 1 or 2, wherein the pharmaceutical composition comprises about 0.01-20% (w/v) bark resin extract from the plant of genus Vismia and about 0.01-20% w/v bark resin extract from the plant of genus Calycophyllum.

4. The pharmaceutical composition of any one of claims 1-3, wherein the pharmaceutical composition comprises about 0.01-10% (w/v) bark resin extract from the plant of genus Vismia and about 0.01-10% w/v bark resin extract from the plant of genus Calycophyllum.

5. The pharmaceutical composition of any one of claims 1-4, wherein ratio of the bark resin extract from the plant of genus Vismia to the bark resin extract from the plant of genus Calycophyllum is 1:1.

6. The pharmaceutical composition of any one of claims 1-4, wherein ratio of the bark resin extract from the plant of genus Vismia to the bark resin extract from the plant of genus Calycophyllum is more than 1:1.

7. The pharmaceutical composition of claim 6, wherein the ratio of the bark resin extract from the plant of genus Vismia to the bark resin extract from the plant of genus Calycophyllum is 5:1 or more.

8. The pharmaceutical composition of claim 7, wherein the ratio of the bark resin extract from the plant of genus Vismia to the bark resin extract from the plant of genus Calycophyllum is 10:1.

9. The pharmaceutical composition of any one of claims 1-4, wherein ratio of the bark resin extract from the plant of genus Vismia to the bark resin extract from the plant of genus Calycophyllum is less than 1:1.

10. The pharmaceutical composition of claim 9, wherein the ratio of the bark resin extract from the plant of genus Vismia to the bark resin extract from the plant of genus Calycophyllum is 1:5 or less.

11. The pharmaceutical composition of claim 10, wherein the ratio of the bark resin extract from the plant of genus Vismia to the bark resin extract from the plant of genus Calycophyllum is 1:10.

12. The pharmaceutical composition of any one of claims 1-11, wherein the bark resin extract from the plant of genus Vismia and the bark resin extract from the plant of genus Calycophyllum are aqueous extracts.

13. The pharmaceutical composition of any one of claims 1-12, wherein the pharmaceutical composition comprises at least 50% (v/v) water.

14. The pharmaceutical composition of any one of claims 1-13, wherein the plant of genus Vismia is Vismia angusta.

15. The pharmaceutical composition of any one of claims 1-14, wherein the plant of genus Calycophyllum is Calycophyllum spruceanum.

16. The pharmaceutical composition of any one of claims 1-15, further comprising a pharmaceutically acceptable carrier, excipient, or vehicle.

17. The pharmaceutical composition of any one of claims 1-16, further comprising one or more antibiotic agents.

18. The pharmaceutical composition of any one of claims 1-17, further comprising one or more antiseptic agents.

19. The pharmaceutical composition of any one of claims 1-18, further comprising one or more antifungal agents.

20. The pharmaceutical composition of any one of claims 1-19, further comprising one or more antiviral agents.

21. The pharmaceutical composition of any one of claims 1-20, further comprising one or more analgesic agents.

22. The pharmaceutical composition of any one of claims 1-21, wherein the pharmaceutical composition is formulated as a topical formulation.

23. The pharmaceutical composition of any one of claims 1-22, further comprising one or more UV-absorbing agents.

24. The pharmaceutical composition of any one of claims 1-23, further comprising one or more moisturizing agents.

25. The pharmaceutical composition of any one of claims 1-24, wherein the pharmaceutical composition is formulated as a lotion, cream, salve, liniment, ointment, gel, paste, tonic, unguent, spray, soap, shampoo, or lip balm.

26. A method of treating a wound in a subject in need thereof, the method comprising administering to the subject a therapeutically effective amount of the pharmaceutical composition of any one of claims 1-25.

27. The method of claim 26, wherein the wound is a chronic wound.

28. A method of treating a chronic wound in a subject in need thereof, the method comprising administering to the subject a therapeutically effective amount of the pharmaceutical composition of any one of claims 1-8 or 12-25.

29. The method of claim 27 or 28, wherein the chronic wound is a venous ulcer, a diabetic ulcer, a pressure ulcer, or rash associated with shingles, psoriasis, allergy, or dermatitis.

30. The method of claim 26, wherein the wound is an acute wound.

31. A method of treating an acute wound in a subject in need thereof, the method comprising administering to the subject a therapeutically effective amount of the pharmaceutical composition of any one of claims 1-5 or 9-25.

32. The method of claim 30 or 31, wherein the acute wound is a trauma wound, a burn wound, or a surgical wound.

33. The method of any one of claims 26-32, wherein the pharmaceutical composition is administered to the subject topically.

34. The method of any one of claims 26-33, wherein the pharmaceutical composition is administered to the subject as a lotion, cream, salve, liniment, ointment, gel, paste, tonic, unguent, spray, soap, shampoo, or lip balm.

35. The method of any one of claims 26-34, wherein the pharmaceutical composition is administered to the subject for 15 days or less.

36. The method of claim 35, wherein the pharmaceutical composition is administered to the subject for 10 days or less.

37. The method of claim 36, wherein the pharmaceutical composition is administered to the subject for 7 days or less.

38. The method of any one of claims 26-37, wherein the pharmaceutical composition reduces inflammation.

39. The method of any one of claims 26-38, wherein the pharmaceutical composition induces wound contraction.

40. The method of any one of claims 26-39, wherein the pharmaceutical composition induces wound closure.

41. The method of any one of claims 26-40, wherein the pharmaceutical composition reduces release of TNF-α from peripheral blood mononuclear cells.

42. The method of any one of claims 26-41, wherein the pharmaceutical composition induces migration of epidermal keratinocytes.

43. The method of any one of claims 26-42, wherein the subject is a human.

44. The pharmaceutical composition of any one of claims 1-25 for use in treating a wound in a subject in need thereof.

45. The pharmaceutical composition for use according to claim 44, wherein the wound is a chronic wound.

46. The pharmaceutical composition for use according to claim 45, wherein the chronic wound is a venous ulcer, a diabetic ulcer, a pressure ulcer, or rash associated with shingles, psoriasis, allergy, or dermatitis.

47. The pharmaceutical composition for use according to claim 44, wherein the wound is an acute wound.

48. The pharmaceutical composition for use according to claim 47, wherein the acute wound is a trauma wound, a burn wound, or a surgical wound.

49. The pharmaceutical composition for use according to any one of claims 44-48, wherein the subject is a human.
